(19) United States
(12) Patent Application Publication
(10) Pub. No.: US 2006/0153918 A1
(43) Pub. Date: Jul. 13, 2006

(54) Dosage forms with an enterically coated core tablet

(76) Inventors: E. Itzhak Lerner, Petach Tikva (IL); Vered Rosenberger, Modiin (IL); Ofer Aqua, Ofra (IL); Moshe Flashner-Barak, Petach Tikva (IL)

Correspondence Address: KENYON & KENYON LLP, ONE BROADWAY, NEW YORK, NY, 10004, US

(21) Appl. No.: 11/190,766

(22) Filed: Jul. 26, 2005

Related U.S. Application Data
(60) Provisional application No. 60/591,482, filed on Jul. 26, 2004.
(60) Provisional application No. 60/591,820, filed on Jul. 27, 2004.

(51) Int. Cl. A61K 9/24

(52) U.S. Cl. 424/473

(57) ABSTRACT

The present invention provides a pharmaceutical dosage form for oral administration to a patient comprising an enterically coated core tablet sheathed in an annular body of compressed powder or granular material. The present invention also provides a pharmaceutical dosage form for co-administration of two or more active pharmaceutical ingredients. The present invention also provides a method comprising administering the dosage form of the present invention to a patient with impaired gastric motility, such as a patient with Parkinson's disease.

This application claims the benefit of U.S. Provisional Patent Application Ser. Nos. 60/591,482 filed Jul. 26, 2004 and 60/591,820 filed Jul. 27, 2004.

### FIELD OF THE INVENTION

The present invention relates to oral pharmaceutical dosage forms and more particularly to forms with an enterically coated core tablet.

### BACKGROUND OF THE INVENTION

Tailoring drug delivery to the needs of therapy is a current goal in the development of drug delivery systems. For some therapies, a controlled release delivery profile is desirable.

Certain axioms guide the development of controlled release drug delivery systems. One such axiom is that ‘flatter is better,’ i.e., the flatter the delivery curve is vs. time, the better the system will behave. It is therefore considered desirable to have delivery systems that give essentially a zero order release profile. The amount of drug released is not dependent on the amount left within the delivery system and remains constant over the entire delivery profile. Tailoring the drug delivery to the needs of the therapy is another axiom of delivery improvement. One can conceive of therapies that need a sudden burst of drug after several hours of constant delivery or a change in the rate of drug delivery after several hours.

There is a need for versatile solid dosage forms.

### SUMMARY OF THE INVENTION

In one embodiment, the present invention provides a pharmaceutical dosage form for oral administration to a patient comprising an enterically coated core tablet containing an active pharmaceutical ingredient sheathed in an annular body of compressed powder or granular material. Preferred active pharmaceutical ingredients include, but are not limited to methylphenidate, rasagiline, carbidopa and levodopa. The core tablet may further contain one or more excipients including, but not limited to anhydrous lactose, hydroxypropylcellulose, microcrystalline cellulose, hydroxypropylmethylcellulose, and crospovidone. The annular body may further contain one or more excipients including, but not limited to polyvinylpyrollidone, microcrystalline cellulose, polyethylene oxide, and ethylcellulose. Preferably, the enteric coating prevents release of the active pharmaceutical ingredient in the stomach and allows release of the active pharmaceutical ingredient in the small intestine.

The release of one or more active ingredients from the dosage form can be measured in a United States Pharmacopeia (USP) standard apparatus II tester in either 900 ml or 500 ml of 0.1 N HCl at 37° C. with a stirring rate of 50 revolutions per minute (rpm).

In another preferred embodiment, the present invention provides a method comprising administering a dosage form of the present invention to a patient with impaired gastric motility. A patient with impaired gastric motility can be, for example, a patient with Parkinson's disease. In this embodiment, the active ingredient is preferably rasagiline.

In yet another preferred embodiment, the present invention provides a pharmaceutical dosage form for co-administration of two or more active pharmaceutical ingredients to a patient comprising an enterically coated core table containing one or more core active pharmaceutical ingredients sheathed in an annular body of compressed powder or granular material and containing one or more annular active pharmaceutical ingredients. Preferably, the core active pharmaceutical ingredient is methylphenidate. Preferably, the annular body contains both carbidopa and levodopa. Preferably, the one or more core active pharmaceutical ingredients are released in the small intestine and the one or more annular active pharmaceutical ingredients are released in the stomach

### BRIEF DESCRIPTION OF THE FIGURES

FIG. 1 shows sectional perspective, side and top down views of a solid dosage form with a recessed core tablet of active ingredient in a compressed annular body of powder or granular material in accordance with the invention.

FIG. 2 is a plot of the average rate of alendronate excretion in urine of humans who had taken a dosage form in accordance with the present invention containing 70 mg monosodium alendronate and a prior art 70 mg monosodium alendronate dosage form.

FIG. 3 is a plot of the rate of release of oxybutynin from a dosage form in accordance with the invention, wherein the rate of release is maintained between 3% h⁻¹ and 12% h⁻¹ for seven hours or more.

FIG. 4 is a plot of the rate of release of oxybutynin from a dosage form in accordance with the invention. The proportion of hydrogel in the core tablet is increased relative to the dosage form that produced FIG. 3 resulting in a decreased maximum rate of release and an extended release between 3% and 12% per hour for about twelve hours.

FIG. 5 is a plot of the rate of release of oxybutynin from a dosage form in accordance with the invention. The proportion of release-inhibiting hydrogel in the annular body was increased relative to the dosage form that produced FIG. 4. The maximum rate of release was further reduced to less than 7% h⁻¹.

FIG. 6 is a plot of the rate of release of carbidopa from the core tablet and of levodopa from the annular body of a dosage form in accordance with the present invention. The core tablet is cylindrically shaped and annular having a 2.5 mm diameter hole therethrough.

FIG. 7 is a plot of the rate of release of carbidopa from the core tablet and of levodopa from the annular body of a dosage form in accordance with the present invention. The core tablet of this dosage form has a 4.6 mm hole, larger than that in the dosage form that produced FIG. 6, resulting in greater surface area and a more rapid rate of release of carbidopa.

FIG. 8 is a plot of the rate of release of carbidopa from the core tablet and of levodopa from the annular body of a dosage form in accordance with the present invention. The dosage form that produced this figure had an oval core tablet with a 3 mm hole therethrough which resulted in a release similar to the cylindrical core table with a 2.5 mm hole (FIG. 6).

### DESCRIPTION OF THE PREFERRED EMBODIMENTS

The novel pharmaceutical dosage form of the present invention comprises a core tablet containing an active pharmaceutical ingredient sheathed in an annular body comprised of compressed powder or granular material. The core tablet has first and second opposed surfaces and a circumferential surface. “Sheathing” means that the annular body encircles the core tablet and is in contact with the core tablet about its circumferential surface, but leaves opposed surfaces of the core tablet substantially exposed. The core tablet contains at least one active pharmaceutical ingredient, but otherwise its formulation is not critical to the invention. The core tablet can be formulated for various release profiles, such as delayed release, burst or pulsed release, sustained or zero order release. The annular body can be formulated to achieve any of a variety of purposes, such as gastric retention, ease of swallowing, taste masking, and controlled rate of drug release from the core tablet. The annular body can contain or be coated with a co-active ingredient.

The pharmaceutical dosage forms of the present invention can be made using the methods and apparatus described in published U.S. patent applications Ser. No. 10/379,338, (Publication No. US 2004-0052843), and Ser. No. 10/419,536, Publication No. US 2003-0206954); both of which are incorporated by reference in their entirety.

The terms “drug” and “active pharmaceutical ingredient” broadly include any biologically, physiologically, or pharmacologically active agent. Active pharmaceutical ingredients that can be administered in the dosage form of the present invention include adrenergic receptor agonists and antagonists; muscarinic receptor agonists and antagonists; anticholinesterase agents; neuromuscular blocking agents; ganglionic blocking and stimulating agents; sympathomimetic drugs; serotonin receptor agonists and antagonists; central nervous system active drugs such as psychotropic drugs, central nervous system stimulants, antipsychotic drugs, antianxiety drugs, antidepressents, antimanic drugs, anesthetics, hypnotics, sedatives, hallucinogenic drugs and antihallucinogenic drugs; antiepileptic drugs; antimigraine drugs; drugs for treatment of Parkinson's, Alzheimer's and Huntington's disease; monoamine oxidase (MAO) inhibitors; analgesics; antitussive agents; antihistaminic drugs; H₁, H₂, and H₃ receptor antagonists; bradykinin receptor antagonists; antipyretic agents; antiinflammatory agents; NSAIDs; diuretics; inhibitors of Na⁺-Cl⁻symport; vasopressin receptor agonists and antagonists; ACE inhibitors; angiotensin II receptor antagonists; renin inhibitors; calcium channel blockers; ∃-adrenergic receptor antagonists; antiplatelet agents; antithrombic agents; antihypertensive agents; vasodialators; phosphodiesterase inhibitors; antiarrhythmic drugs; HMG CoA reductase inhibitors; H⁺, K⁺-ATPase inhibitors; prostaglandins and prostaglandin analogs; laxatives; antidiarrheal agents; antiemetic agents; prokinetic agents; antiparasitic agents such as antimalarial agents, antibacterial agents, drugs for treatment of protozoal infections and antihelmintic drugs; antimicrobial drugs such as sulfonamides, quinolones, β-lactam antibiotics, aminoglycosides, tetracyclines, chloramphenicol and erythromycin; drugs for treatment of tuberculosis, drugs for treatment of leprosy; antifungal agents; antiviral agents; antineoplastic agents; immunomodulators; hematopoietic agents; growth factors; vitamins; minerals; anticoagulants; hormones and hormone antagonists such as antithyroid drugs, estrogens, progestins, androgens, adrenocortical steroids and adrenocortical steroid inhibitors; insulin; hypoglycemic agents; calcium resorption inhibitors; clucocorticoids; retinoids and heavy-metal antagonists.

Preferred active pharmaceutical ingredients include, but are not limited to alendronate monohydrate, monosodium alendronate trihydrate, sodium etidronate, sodium risedronate, pamidronate, aspirin, ibuprofen, naproxen, fenoprofen, ketoprofen, oxaprozin, flubiprofen, indomethacin, sulindac, etodolac, mefenamic acid, meclofenamate sodium, tolmetin, ketorolac, diclofenac, piroxicam, meloxicam, tenoxicam, phenylbutazone, oxyphenbutazone, oxybutynin, alendronate, carbidopa, levodopa, methylphenidate, rasagiline, tizanide, sumatriptan, pharmaceutically acceptable salts, hydrates, isomers, esters and ethers thereof, and mixtures thereof.

The annular body can be formed of any powdered or granular pharmaceutically acceptable excipients and can itself include an active pharmaceutical ingredient. In particular, the annular body can include diluents, binders, disintegrants, glidants, lubricants, flavorants, colorants and the like. Powdering and granulation with conventional excipients and the techniques for forming compressed bodies therefrom with given characteristics in terms of friability, hardness and freedom from capping is well within the knowledge of those skilled in the art of tableting.

Preferred excipients for forming the annular body include hydroxypropyl cellulose (e.g., Klucel™), hydroxypropyl methylcellulose (e.g. Methocel™), microcrystalline cellulose (e.g., Avicel™), starch, lactose, sugars, polyvinylpyrrolidone (e.g., Kollidon™, Plasdone™), calcium phosphate, and MicrocelLac100™ (a 25:75 mixture of microcrystalline cellulose and lactose).

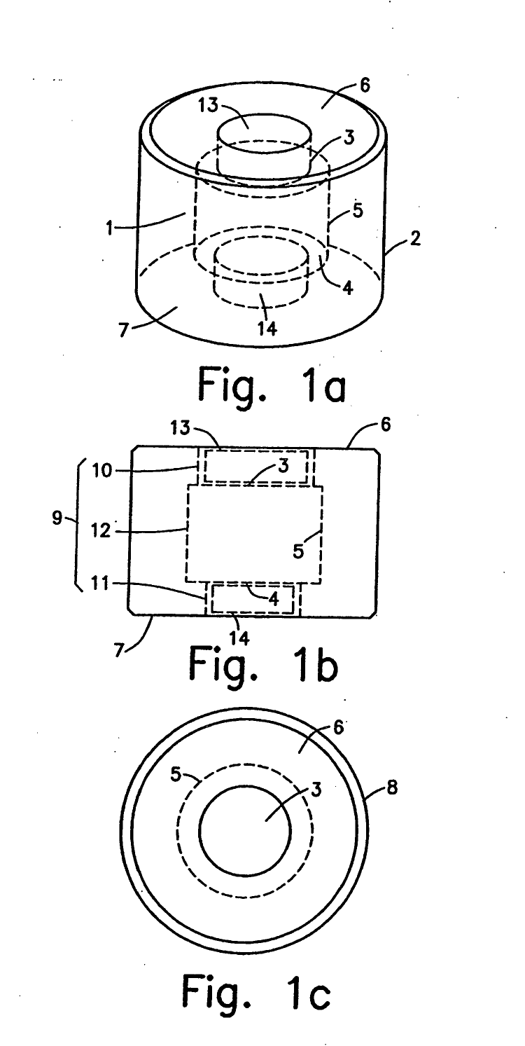

In the embodiment illustrated in FIG. 1, core tablet 1 containing the active pharmaceutical ingredient is recessed in the annular body 2, which is composed of non-ulcerative pharmaceutical excipients. The “recessed” tablet is especially well suited for oral delivery of ulcerative drugs. It reduces the incidence of pill esophagitis and contact gastritis by localizing the ulcerative drug in a core tablet that is shielded from contact with the mucosa lining the gastrointestinal tract. Recessing the core tablet does not significantly alter the release profile of the core tablet because a sizable portion of the surface of the core tablet is in fluid communication with the environment.

Both the core tablet and the annular body may be formed into any suitable shape. Specific shapes can be achieved by use of specifically designed punches. Preferably the core tablet and the annular body are cylindrical in shape. The core tablet and the annular body may be the same or different in shape. The exposed surfaces of the core tablet may be of any suitable shape. Preferably, the exposed surfaces of the core tablet are circular or oval.

Turning again to FIG. 1, core tablet 1 has opposed first and second surfaces 3 and 4 and an outer circumferential surface 5 extending between the opposed surfaces. Core tablet 1 is preferably cylindrical or disk shaped for ease of manufacture, but need not be so. In a dosage form for administration to humans, the maximum distance across either of the opposed surfaces 3 or 4 is preferably from about 2 mm to about 12 mm, more preferably from about 4 mm to about 7 mm, most preferably about 5 mm. Opposed surfaces 3 and 4 can be flat, concave or convex and are preferably flat for bearing modest axial compression forces exerted by flat pressing surfaces during formation of the annular body about the core tablet.

In outer contour, annular body 2 is preferably cylindrically shaped, but it can have any cross-section, such as oval, elliptical or oblong. The outer diameter is preferably of from about 5 mm to about 15 mm, more preferably of from about 7 mm to about 12 mm, most preferably about 9 mm. The inner diameter can be any size up to about 2 mm less than the outer diameter. A narrow inner diameter less than 2 mm may slow release of the drug if an excipient in the annular body swells upon contact with gastric fluid. However, in some embodiments, a lower limit 0.5 mm may still be useful. Preferably, the inner diameter is 3 mm or greater.

Annular body 2 has opposed first and second annular faces 6 and 7, an outer circumferential surface 8 extending between the annular faces from their outer edges, and an inner circumferential surface 9 extending between the annular surfaces from their inner edges, thus defining an annulus.

As best seen in side view (FIG. 1B), inner circumferential surface 9 of annular body 2 consists of three longitudinal (axial) segments. First and second segments 10 and 11 are terminal and do not contact the sides of the core tablet. They are separated by an internal third segment 12 that contacts the outer circumferential surface 5 of core tablet 1. Opposed surfaces 3 and 4 of the core tablet are therefore recessed from annular faces 6 and 7 of the annular body. Opposed surfaces 3 and 4 are preferably recessed from about 0.5 mm to about 4 mm, more preferably about 1.5 mm relative to the annular faces 6 and 7 of the annular body (said recessed distance corresponding to the length of the corresponding terminal segment). The recess depth of surfaces 3 and 4 can be the same or it can be different.

By recessing the drug-containing core tablet, potential damage to the mucosa caused by contact of an ulcerative active agent with the mucosa can be mitigated since any contact between the dosage form and the mucosa is with a surface of the annular body made of non-ulcerative components. However, one or both of opposed surfaces 3 and 4 can be flush with annular faces 6 and 7 of the annular body without deleterious effect when the dosage form of the present invention is used to administer non-ulcerative drugs or when the core tablet is otherwise protected, such as by a coating.

To better apprehend the recessed dosage form embodiment of the invention, it is useful to conceive of surface 3 of the core tablet and first longitudinal segment 10 as defining a first void 13. Likewise, surface 4 of the core tablet and second longitudinal segment 11 define a second void 14. Voids 13 and 14 fill with gastric fluid when the dosage form is immersed in gastric fluid after reaching the stomach. Gastric fluid passes through the voids to contact the core tablet and the drug leaves through the voids after it is dissolved. Voids 13 and 14 are preferably from about 0.5 mm to about 10 mm, more preferably from about 3 mm to about 6 mm and most preferably about 4.5 mm in width (measured parallel to first or second opposed surfaces). Drug release, therefore, does not occur by an osmotic mechanism such as occurs with pierced dosage forms made using the apparatus of U.S. Pat. No. 5,071,607.

Opposed surfaces 3 and 4 of the core tablet are preferably substantially exposed, i.e., they are not substantially covered by the annular body. “Substantially exposed” means that less than about 50% of each of the opposed surfaces is concealed or hidden from visual inspection by the annular body. Such differences may result in inner segment 12 being offset from terminal segments 10 and 11, which, themselves, can have different longitudinal cross sections, e.g., have different diameters, as depicted in FIG. 1. Alternatively, the cross section of the annulus defined by inner circumferential surface 9 can be uniform throughout its length. Although a portion of opposed surfaces 3 and 4 can be concealed by the annular body that is not necessarily the case.

Further, the invention contemplates that the rate of release of the drug is determined by the formulation and shape of the core tablet, not by diffusion of the drug through the annular body, which contributes to the versatility of the dosage form for different release profiles. The core tablet can be formulated for immediate or controlled release, including sustained release and delayed release. In preferred embodiments of this invention, the core tablet is enterically coated.

In one embodiment, the pharmaceutical dosage form is an extended release dosage form. Active drug material is delivered via the exposed axial surfaces of the core tablet. The exposed axial surfaces retain a constant cross-section during delivery of the active material, thus producing a zero-order release profile. For extended release applications, the core tablet can be formulated to be of an eroding or diffusive nature.

An extended release core tablet preferably contains a hydrogel such as hydroxypropyl methylcellulose, hydroxypropyl cellulose, ethylcellulose and the like. Optionally, the core tablet also contains a more rapidly dissolving substance like compressible sucrose to open pores in the hydrogel matrix and thereby modulate the hydrogel grip on the active ingredient. In a zero order extended release dosage form wherein the active ingredient is contained in the core tablet, the annular body will be formulated to be yet slower dissolving than the core tablet so that the surface area of the core tablet will remain constant. Mixtures of about 1 part high molecular weight polyethylene glycol (PEG) and 3-5 parts ethyl cellulose will retain their shape and rigidity in water for the time that it takes for most conventional eroding or swelling hydrogel matrices to completely release the drug. An especially preferred composition of the annular body of an extended release dosage form in accordance with this invention comprises about 15-25 parts PEG 4000, about 70-80 parts ethylcellulose and about 5 parts polyvinylpyrrolidone. The rate of release of active material from the core tablet of extended release dosage forms is less than about 15% by weight per hour. Preferably the rate of release is from about 3% per hour to about 12% by weight per hour. Extended release dosage forms are adapted for the release of active material over a period of at least about 4 hours, more preferably at least about 7 hours, and most preferably at least about 10 hours. The rate of release of active ingredient is measured in a United States Pharmacopeia standard apparatus II solution tester in an aqueous solution buffered at 6.8 at 37° C. with a stirring rate of 50 revolutions per minute.

The core tablet also can be a bilayer tablet with each layer containing the same or different drugs and each layer releasing the drug at the same or at different rates. One of the layers could be an immediate release layer and the other a slow release layer, or both can be slow release layers. The core tablet can be formulated to be a three layer tablet with the central layer being a drug to be delivered after a delay. The two outer layers can be delay layers or drug delivery layers with the same or different drugs and with the same or different release profiles. The middle layer can contain again the same or different drugs compared to the outer layers and can be of a controlled release or an immediate release nature. Thus, one can have controlled release of two drugs each at its optimal release rate and a delayed release or delayed pulse of a third drug. The currently described invention thus gives a very wide range of drug delivery capabilities not addressed by conventional dosage forms and improves upon the performance of other known delivery systems.

Dosage forms in accordance with the invention also can be formulated to deliver more than one active pharmaceutical ingredient by locating one or more active pharmaceutical ingredients in the core tablet and one or more active pharmaceutical ingredients in the annular body. Such an arrangement enables the release rate of each active ingredient to be controlled independently by formulation adjustments to the portion of the dosage form, i.e. core tablet or annular body, that contains the drug that is being released either too slowly or too quickly. In addition, the shape of one of the portions can be changed without adjusting the formulation. For instance, the powder or granular material may be pressed around the core tablet into a body having an oval cross-section rather than a circular cross-section to achieve a faster rate of release (resulting from increased surface area). In addition, the core tablet may have a hole extending from one axial face to the other in order to increase the surface and thereby increase the release rate. The release rate can be further controlled through changes to the diameter of the hole.

In a preferred embodiment, the pharmaceutical dosage form of this invention has a core tablet that is enterically coated before it is sheathed in the annular body. The enteric coating of the present invention can be any enteric coating known in the art, e.g., EUDRAGIT®, EUDRAGIT®S, and cellulose acetate phthalate. Such enteric coating materials are pH-sensitive and can withstand prolonged contact with acidic gastric fluids. Therefore, the enteric coating does not dissolve until after stomach passage but dissolves readily in the mildly acidic to neutral environment of the small intestine. The level of coating necessary to achieve the desired delay of onset of drug release can be readily determined by experimentation of one skilled in the art (see, e.g., United States Pharmacopeia, 26^{th} Rev./National Formulary, 21^{st} Ed., 2002, <724> Drug Release, Delayed-Release (Enteric-Coated) Articles—General Drug Release Standard, 2160-2161; Pharmaceutical Dosage Forms and Drug Delivery Systems, H. C. Ansel, L. V. Allen, Jr., N. G. Popovich (Lippincott Williams & Wilkins, pub., 1999), Modified-Release Dosage Forms and Drug Delivery Systems, 223, 231-240).

Preferably, the enteric coating prevents release of the active pharmaceutical ingredient in the stomach and allows release of the active pharmaceutical ingredient in the small intestine. Thus, the enterically coated core tablet is useful to administer drugs that are preferably released in the small intestine. Also, the enterically coated core tablet is useful to administer drugs that are preferably not released in the stomach. For instance, when the active pharmaceutical ingredient is an ulcerative drug, the enteric coating shields the drug from the gastrointestinal mucosa.

As described above, each of the core tablet and annular body may contain one or more active pharmaceutical ingredients. For co-administration of more than one active pharmaceutical ingredient, the enteric coating allows a drug delivery system wherein a core active pharmaceutical ingredient contained in the core tablet is released in the small intestine, and an annular active pharmaceutical ingredient contained in the annular body is released in the stomach. In this embodiment, the annular active pharmaceutical ingredient begins to be released in the acidic conditions of the stomach, while the core active pharmaceutical ingredient is protected by the enteric coating. Then, after the dosage form passes into the small intestine, the core active pharmaceutical ingredient is released. Each of the core tablet and the annular body may be independently formulated to release the active pharmaceutical ingredient(s) therein in an immediate or a controlled manner.

In a preferred embodiment, the annular body is formulated for the gastric release of both levodopa and carbidopa, and the core tablet is formulated for the delayed release of methylphenidate. This embodiment is useful for the improved treatment of Parkinson's disease as described in U.S. Provisional Patent Application 60/512,973, incorporated herein by reference.

When the core tablet is enterically coated, the dosage form is particularly useful for administration to a patient with impaired gastric motility. In certain diseases, such as Parkinson's disease, it is well known that many patients suffer from delayed gastric emptying. (See e.g., R. F. Pfeiffer & E. M. M. Quigley, Gastrointestinal motility problems in patients with Parkinson's disease: Epidemiology, pathophysiology, and guidelinesfor management, CNS-Drugs 11(6): 435-448 (1999); W. H. Jost, Gastrointestinal motility problems in patients with Parkinson's disease: Effects of antiparkinsonian treatment and guidelines for management, Drugs and Aging, 10(4): 249-258 (1997)). In patients with impaired gastric motility, a dosage form can be delayed from leaving the stomach (delayed gastric emptying) or the dosage form stays in the stomach longer than usual (prolonged gastric residence). An enteric coating can fail due to delayed gastric emptying and/or prolonged gastric residence. The enteric coating is weakened by extended exposure to gastric acids, especially in combination with the mechanical forces of natural stomach churning. Under these conditions, the enteric coating may leak or fail completely. The partial or complete failure of an enteric coating can cause catastrophic consequences because it leaks or dumps a drug into the stomach that is not meant to be released into the stomach. Consequences include inactivation of the drug by gastric acids or considerable morbidity. Because the enterically coated core tablet of the present invention is sheathed in an annular body, the enterically coated core tablet is protected against the mechanical forces of the stomach. Thus, the present invention helps to minimize the failure of the enteric coating.

In another preferred embodiment, the enterically coated core tablet of the present invention is used to administer rasagiline to patients with Parkinson's disease. Rasagiline is a monoamine oxidase (MAO) inhibitor that crosses the blood brain barrier. Rasagiline allows the brain to better utilize dopamine by preventing the destructive metabolism of the dopamine. To minimize peripheral inhibition of MAO, rasagiline is preferably released after passage through the stomach. When rasagiline is administered in a simple enterically coated dosage form, the enteric coating may fail because of the impaired gastric motility associated with Parkinson's patients. The present invention minimizes the threat of enteric coating failure by sheathing the enterically coated core tablet in an annular body.

Having thus described the present invention with reference to certain preferred embodiments, the invention will now be further illustrated by the following non-limiting examples.

### EXAMPLES

### Example 1

### Immediate Release Monosodium Alendronate Tablets

This example summarizes a study designed to determine the rate and extent of absorption of alendronate sodium in human subjects upon administration of a solid pharmaceutical dosage form of the present invention (“protected tablet”).

### Materials and Methods

Protected tablets were made as follows:

Core Tablet: 85.4 g of alendronate trihydrate (TEVA Assia Ltd.) and 2.6 g of xylitol (Danisco Sweeteners OY) were granulated with 20 g water in a Diosna (model P1/6) granulator for 3 min. The granulate was dried at 40° C. for one hour in a fluidized bed dryer and milled through a 0.8 mm screen. The granulate was blended with 11 g crospovidone NF (BASF Pharma) for five minutes. One gram magnesium stearate NF/EP (Mallinkrodt Inc.) was added and the granulate was further blended for an additional 0.5 minutes. The blend was compressed using a Manesty F3 single punch tablet machine fitted with a 5 mm flat beveled punch. The tablet weight was 94.9 mg±1.0% RSD. The hardness of the core tablets was 3-6 kP.

Protected Tablets: A mixture of 94 grams compressible sucrose (Nu-Tab™, DMV International ) and 5 grams microcrystalline cellulose (Avicel™ pH102, FMC International) were blended for five minutes. One gram magnesium stearate (NF/EP, Mallinkrodt Inc.) was added and the mixture was blended for another half a minute.

A Manesty f3 single punch tableting machine was fitted with a spring-biased columnar punch and punch assembly constructed in accordance with the present invention. The core rod was designed for a 5 mm round core tablet and the die and punches for the annular body were designed to produce a round, 9 mm diameter, flat beveled solid pharmaceutical dosage form. The upper punch had a protrusion of diameter 4.5 mm and 1.2 mm height. The tablet press was operated and the protected tablets were produced. The tablet weight was 474 mg±0.62% RSD and the hardness of the protected tablets was 12-15 kP. The alendronate trihydrate content, expressed as alendronic acid was 66.8 mg±1.38% RSD (82.4 mg alendronate trihydrate being equivalent to 70 mg alendronic acid).

The drug-containing core tablet was recessed from the surface of the annular body by about 1 mm.

### Pharmacokinetic Study

A clinical trial involving twelve (12) human volunteers was conducted to demonstrate the pharmacokinetics of a solid dosage form of the present invention containing 70 mg alendronate. Its pharmacokinetics was compared to that of a commercial 70 mg Fosalan™ tablet of the prior art (Merck, Sharpe & Dohme).

### Method

The study was a randomized, open-label, 2-treatment, 2 period, 2 sequence crossover design under fasting conditions. Twelve (12) healthy adult male volunteers, 18-55 years of age were the subjects in the study.

The study was divided into first and second study periods, each of 36 hours duration, with a 14 day “wash-out” period between the study periods. All subjects who completed both study periods were included in the analysis. Subjects were randomly assigned to two groups. One group was administered alendronate via the protected tablet in the first period and administered control Fosalan in the second period. The order of administration to the second group was reversed.

In both periods, alendronate was administered in the fasted state. A standardized meal was provided 4 hours after administration. Snacks were provided on a standardized schedule that was the same for all subjects in both study periods. Water was provided ad libitum. In addition, subjects were encouraged to drink at least 200 ml of water at regular intervals during each study period.

The bioavailability of alendronate was determined by measuring the cumulative levels of alendronate excreted in the urine over a 36 hour period following oral ingestion of the test and control tablets (hereafter “Ae₀₋₃₆”). An initial (t=0) urine sample was taken immediately after administration. Urine samples were taken at 11 regularly scheduled points in time over the 36 hour test period. All urine samples were analyzed for alendronate using a validated HPLC-FLR assay.

### Results

The main pharmacokinetic parameters obtained from the analyses of urine samples are collected in Table 1.

TABLE 1
Pharmacokinetic Parameters

| | Administration via | | | Administration via | | |
|---|---|---|---|---|---|---|
| | Protected Tablet | | | Fosalan (control) | | |
| Parameter | Mean | ± SD | CV (%) | Mean | ± SD | CV (%) |
| Ae₀₋₃₆ (μg) | 113.6 | 77.2 | 67.9 | 102.6 | 36.8 | 36.8 |
| Rₘₐₓ (μg/h) | 37.9 | 19.9 | 51.5 | 31.7 | 11.8 | 38.3 |
| Tₘₐₓ (h) | 1.4 | 0.9 | — | 1.4 | 0.9 | — |

A comparison of the pharmacokinetic parameters of the dosage form in accordance with this invention with the pharmacokinetic parameters of the prior art dosage form is provided in Table 2.

TABLE 2
Comparison of Pharmacokinetics of the Protected Tablet to Prior Art

| | Ae₀₋₃₆ (mg) | Rₘₐₓ (mg/h) |
|---|---|---|
| Geometric Mean of Ratio | 0.99 | 1.12 |
| 90% Geometric C.I. | 75.31% to 128.79% | 93.98% to 135.01% |
| Intra-subject C.V. | 37.48% | 24.85% |

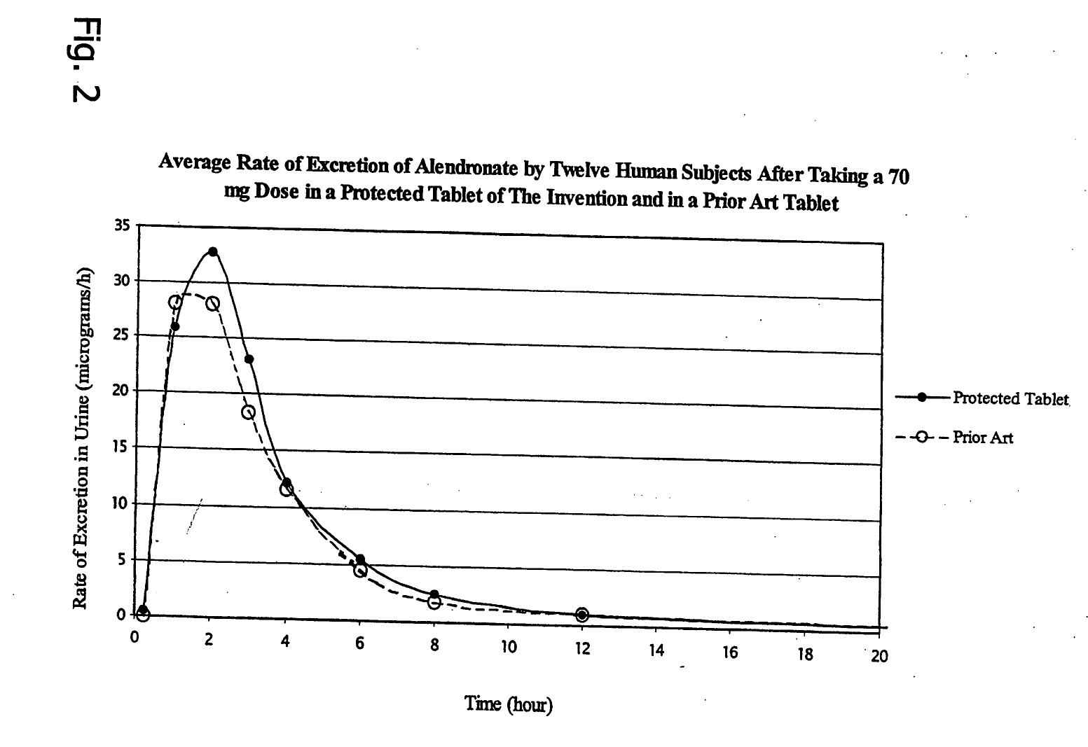

By reference to Tables 1 and 2, and FIG. 2, one can see that alendronate administered via the solid dosage form of the present invention gives essentially the same pharmacokinetic results as administration via Fosalan. The total amount of the alendronate excreted into urine over 36 hours is essentially the same for both treatments with the maximum rates of excretion (parallel to Cₘₐₓ in a pharmacokinetic study of plasma levels of drug) also close.

The profile of excretion into urine was similar for all subjects and in both treatments. The majority of the subjects had their maximum rate of excretion (Rₘₐₓ) between one and two hours. For five of the subjects, the Rₘₐₓ occurred earlier than 1 hour after administration when they took Fosalan. Four of the subjects experienced a Rₘₐₓ in less than an hour when they took the protected tablet. One of the subjects had an Rₘₐₓ in the third hour when he took Fosalan while two of the subjects had a Rₘₐₓ in the third hour when they took the protected tablet.

The total amount of excreted alendronate ranged from 36.9 μg to 158.6 μg when Fosalan was administered and from 30.1 μg to 284.4 μg when the solid oral dosage form of the present invention was administered. In only two subjects was there a greater than two fold difference between the total amount of excreted alendronate between the two treatments. Another subject excreted a very low amount of alendronate regardless of how the alendronate was administered.

The bioavailability of alendronate administered via the novel solid dosage form of the present invention is equivalent to that of alendronate administered by dosage forms of the prior art. However, the dosage form of the prior art does not provide any protection against contact of the alendronate with the mucous membranes of the esophageous and stomach while the bioequivalent novel dosage form of the present invention affords such protection.

### Drug Release Profile

Dissolution was measured in a USP apparatus III dissolution unit (Hanson B-3) unit at 37° C. The alendronate content of samples taken at 5, 10, 15 and 30 minutes was determined by HPLC on an anion column using refractive index detection. The results of the dissolution are reported in Table 3.

TABLE 3

| | Cumulative Percent |
|---|---|
| Time (m) | Release |
| 5 | 48 |
| 10 | 70 |
| 15 | 85 |
| 30 | 98 |

The annular body took more than one hour to dissolve.

The tablets were tested in a human pharmacokinetic study and shown to be bioequivalent to commercially available alendronate (70 mg).

### Example 2

### Extended Release (Zero Order Release) Oxybutynin Tablets

The dosage form of the present invention is uniquely fit for extended controlled release, particularly when one needs to approximate zero order release over an extended period of time. The drug is delivered through the exposed axial faces of the delivery system. These faces retain a constant cross-section during drug delivery, thereby aiding in the achieving of a constant rate of drug release.

### A. Core Tablet

Oxybutynin (50 g), was mixed with anhydrous lactose (50 g) in a Zanchetta Rotolab™ one pot granulator. The granulation solution, 5% w/w hydroxypropylcellulose (Klucel™ LF, 21 ml), was added with stirring at 500 rpm until thorough mixing was achieved. The granulate was dried in the one pot granulator at 45-50° C. with gas stripping for a time of about 20 minutes. The granulate was milled in a Quadro Comil™ milling machine using a screen size of 1143 μm.

The oxybutynin granulate (27.6 g), was mixed with hydroxypropylmethylcellulose, (HPMC, Methocel™ K15M, 19 g), and compressible sucrose (Nu-Tab™, 52.4 g). Magnesium stearate, 1 g, was added with mixing. The blend was compressed into tablets on a Manesty f3 single punch tablet machine using 6 mm flat beveled punches to produce tablets weighing about 110 mg and having a hardness of 4 Kp.

### B. Non Dissolving Annular Body on Cylindrical Surfaces

Polyethylene glycol (PEG 4000) was milled and passed through a 500 μm screen. The milled PEG 4000 (24 g), was mixed with polyvinylpyrrolidone (Povidone™, PVP K-30, 5 g), and ethylcellulose (Ethocel™ 7 cps, 71 g), for 3 minutes. Magnesium stearate (1 g), was added and the blend mixed for another 0.5 minutes. The core tablets, produced above, were pressed within the annular body using this blend and a 9 mm outer cylinder spring loaded core rod tooling previously described. The core rod diameter was 4.5 mm. The upper punch had a protrusion of 5 mm diameter tapering to 4.5 mm at its upper surface with a height of 1.2 mm. The final product, an annular body sheathing a core tablet with recessed exposed axial faces, had an outer diameter of 9 mm, a total weight of 350 mg and contained 15 mg oxybutynin (Formulation A).

### C. Drug Release Profile

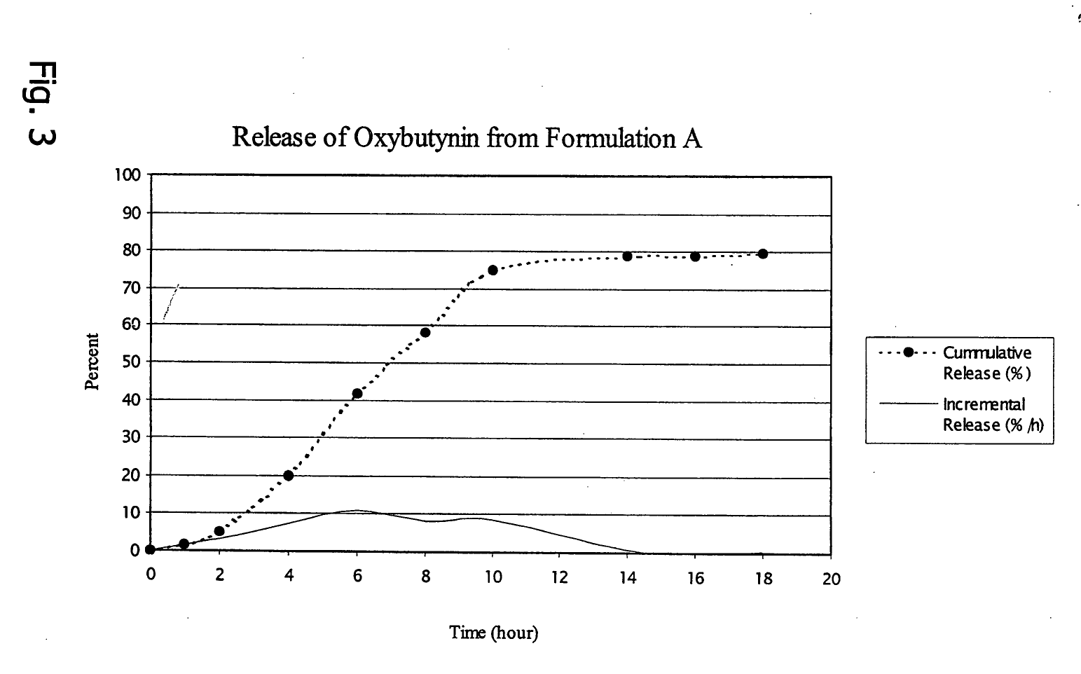

The drug release profile of oxybutynin from the delivery system of Example 1 was tested in an USP apparatus II dissolution tester using 900 ml of phosphate buffer pH=6.8 at 37° C., 50 rpm. The oxybutynin content of the samples were determined by an HPLC method with UV detection. The results are reported in Table 4, below, a presented graphically in FIG. 3.

TABLE 4

| | Cumulative Percent |
|---|---|
| Time (h) | Release |
| 1 | 1.7 |
| 2 | 4.9 |
| 4 | 20.0 |
| 6 | 41.8 |
| 8 | 58.3 |
| 10 | 75.1 |
| 14 | 79.0 |
| 16 | 79.1 |
| 18 | 79.5 |

### D. Control of the Release by Changes in the Core Tablet Formulation

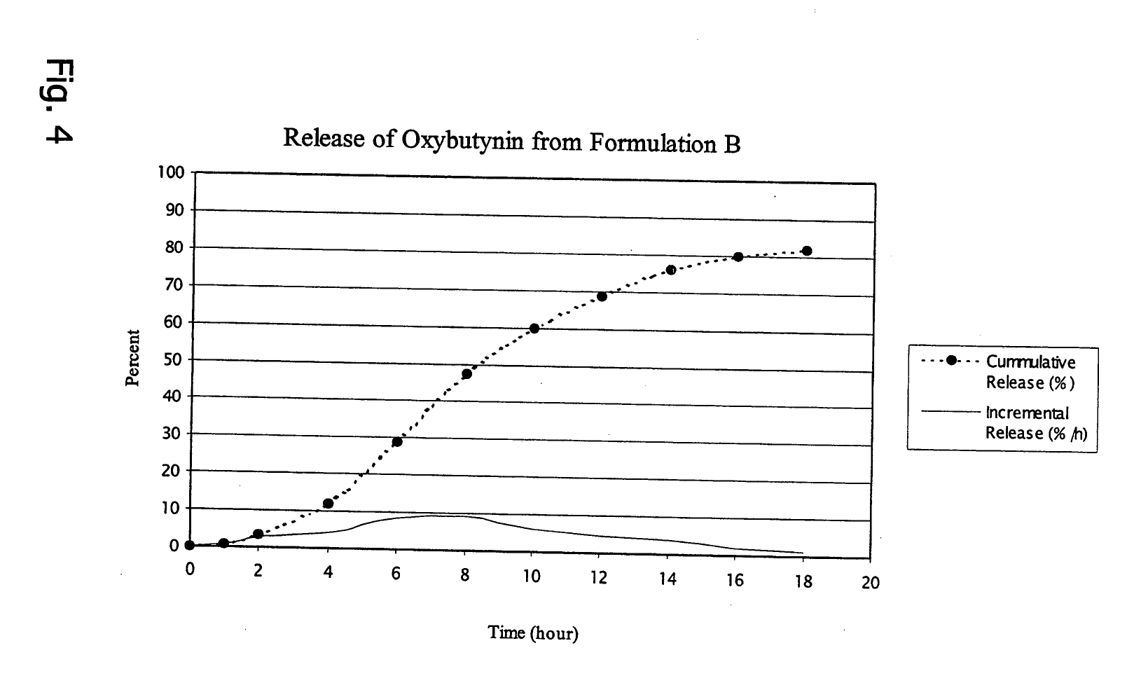

The above procedure for the preparation of the core tablet was repeated, using 30 g of Methocel™ K15M and 41.4 g of Nu-Tab™, thus raising the content of the gel forming HPMC and lowering the content of the dissolving sucrose (Formulation B). The results of the dissolution experiment are reported in Table 5, below, and depicted in FIG. 4.

TABLE 5

| | Cumulative Percent |
|---|---|
| Time (h) | Release |
| 1 | 0.8 |
| 2 | 3.4 |
| 4 | 11.8 |
| 6 | 29.1 |
| 8 | 47.5 |
| 10 | 59.8 |
| 12 | 68.8 |
| 14 | 76.2 |
| 16 | 79.8 |
| 18 | 82.0 |

A significant slowing of drug release in the first ten hours was observed.

### E. Control of Release by Changes in the Formulation of the Annular Body

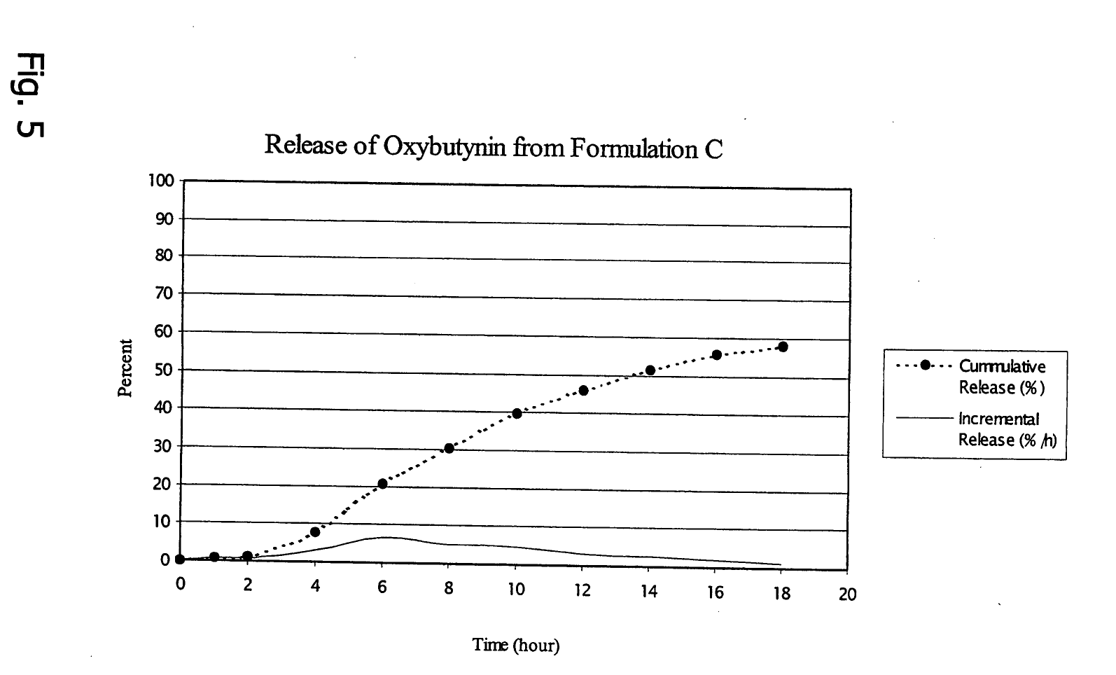

The procedure for the preparation of Formulation B was repeated, with the annular body containing 14 g of PEG 4000 and 81 g of Ethocel™ (Formulation C). The results of the dissolution experiment are shown in Table 6, below, and depicted graphically in FIG. 5.

TABLE 6

| | Cumulative Percent |
|---|---|
| Time (h) | Release |
| 1 | 0.6 |
| 2 | 1.2 |
| 4 | 7.6 |
| 6 | 20.5 |
| 8 | 30.5 |
| 10 | 39.6 |
| 12 | 46.1 |
| 14 | 51.5 |
| 16 | 55.5 |
| 18 | 58.0 |

Again, significant changes in the rate of drug release were observed, demonstrating that changes in the formulation of the core tablet or the annular body can determine the rate of active drug material.

### Example 3

### Release of Two Drugs at Different Rates

The annular body and core tablet can be formulated to contain different drugs and to release the drugs with totally different release profiles. The rates of release can be controlled by the formulation of the core tablet and annular body and by their geometries. In this case, we have formulated a carbidopa immediate release profile in the core tablet with controlled release of levodopa from the annular body while using an oval tablet as the annular body around either a cylindrical or an oval core tablet. The core tablets, both cylindrical and oval, were themselves hollow with a cylindrical hole in each of them.

### A. Core Tablets

Carbidopa (160 g) was mixed with pre sieved (500 μm screen) xylitol (40 g) in a Diosna p1/6 granulator. Water (45 ml) was added as the granulation solution. The mixture was granulated for 5 minutes at 500 rpm and further massed at 800 rpm for 1.5 minutes. The granulate was air dried at room temperature overnight and then milled, while still wet, through a 1.6 mm screen. The milled granulate was dried in a fluidized bed for 30 minutes at 40° C. and then milled through a 0.8 mm screen. This granulate, 56.3 g, was mixed with crospovidone (10 g) and MicrocelLac100™ (32.7 g) for three minutes. Magnesium stearate (1 g) was added to the blend which was further mixed for 0.5 minutes. The blend was compressed in a Manesty f3 single punch tableting machine using three different core rod punches to make hollow cylinders of the following dimensions:

Formulation D: cylindrical outer diameter 7.5 mm inner diameter 2.5 mm

Formulation E: cylindrical outer diameter 7.0 mm inner diameter 4.6 mm

Formulation F: oval outer diameters 12×6 mm, inner diameter 3 mm.

Each tablet contained 54 mg carbidopa.

### B. Drug Containing, Non Dissolving, Oval Annular Body

Levodopa (150 g) was mixed with xylitol (75 g) and hydroxypropylcellulose (Klucel™ LF, 25 g) at 500 rpm for 5 minutes. Ethanol (50 ml) was added slowly and the granulate was formed at 500 rpm over 1.5 minutes. The granulate was air dried overnight at room temperature and milled through a 0.8 mm screen.

The levodopa granulate (44.4 g) was mixed with ethylcellulose (Ethocel™ 7 cps, 30 g) and Cellactose 80™ (25:75 mixture of powdered cellulose: lactose for direct compression, 24.6 g) for three minutes. Magnesium stearate (1 g) was added and the blend mixed for another 0.5 minutes.

The previously formed core tablets, Formulations D, E and F were compressed in an oval shaped annular body core on their radial surfaces using an oval shaped spring loaded core rod punch as previously described, with dimensions 17.6×8.8 mm with an internal core rod of 5 mm diameter and an upper punch with a protrusion of 5 mm diameter tapering to 4.5 mm at its height of 1.8 mm. The total weight of each tablet was 750 mg and each contained 200 mg of levodopa.

### C. Drug Release Profile

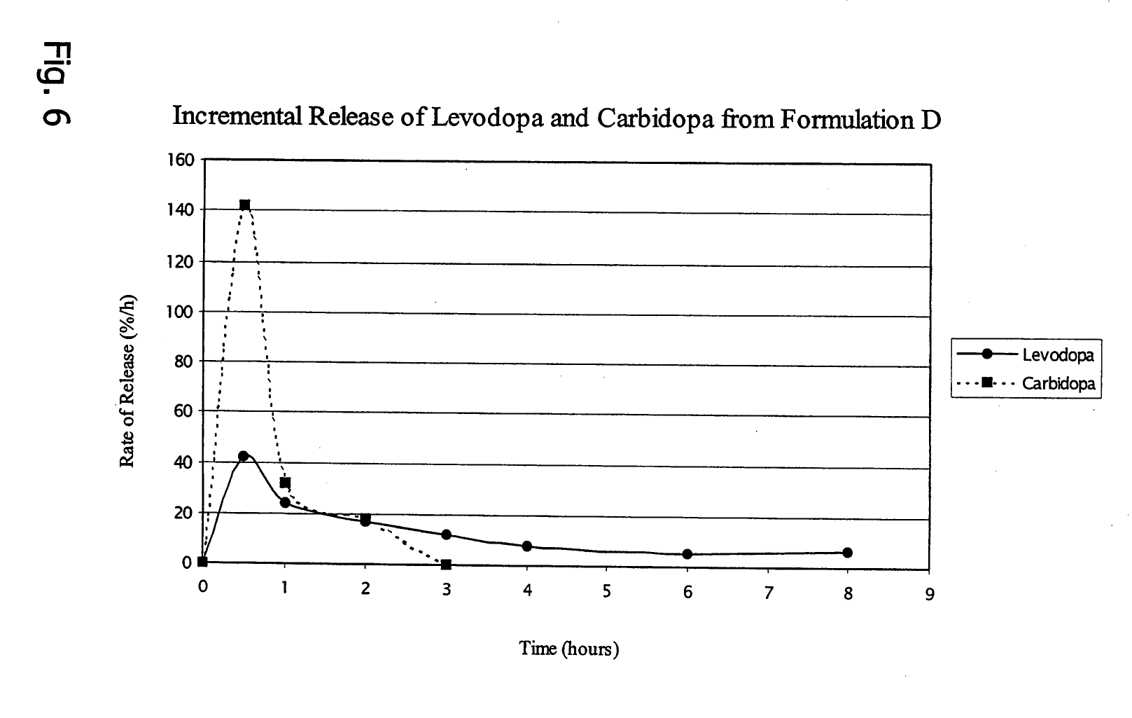

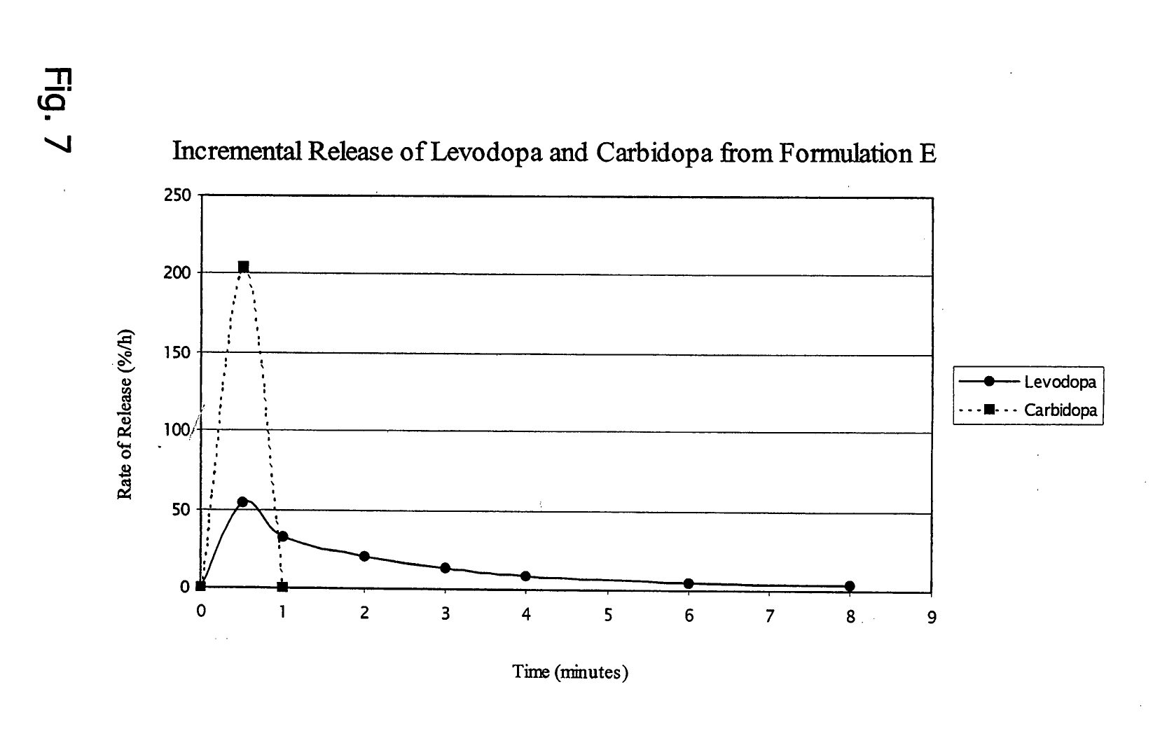

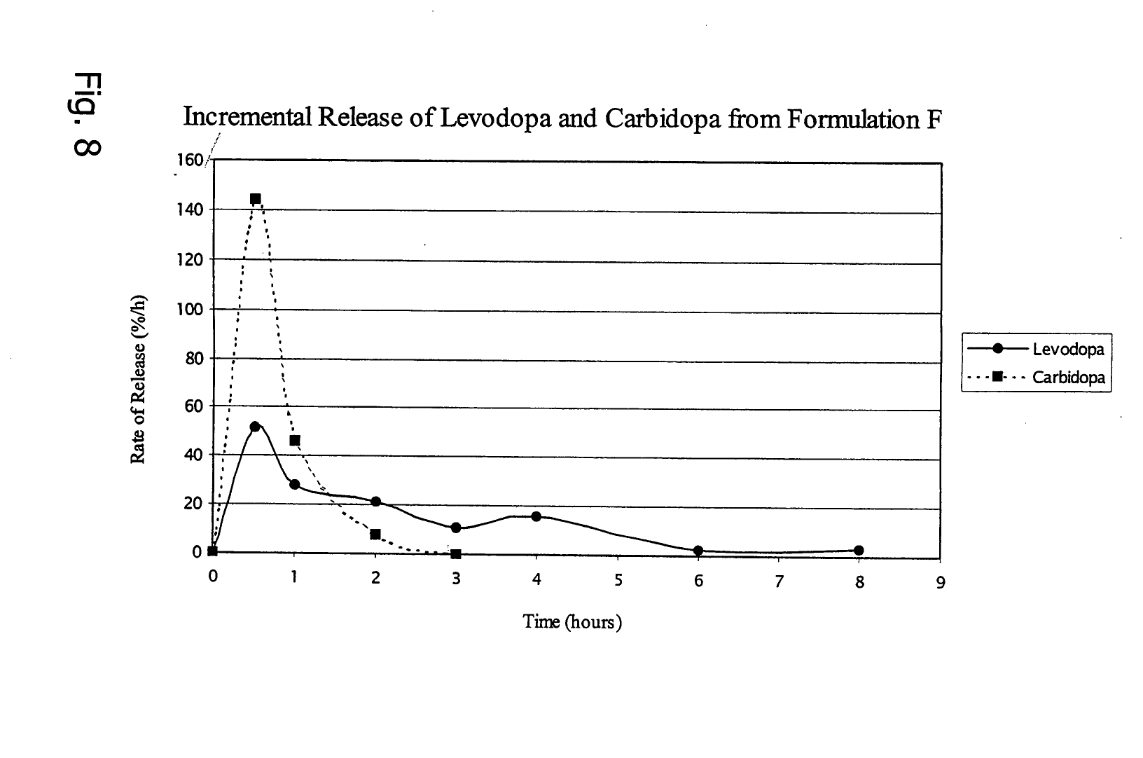

Dissolution was carried out in 0.1N HCl (900 ml) at 37° C. in a USP Apparatus II dissolution tester at 50 rpm and the levodopa and carbidopa concentrations of each sample were determined by HPLC. The results of the dissolution experiments are provided in Tables 7, 8 and 9 and depicted in FIGS. 6, 7, and 8.

TABLE 7
Dissolution Results for Formulation D

Cumulative Percent Release

| Time (h) | Levodopa (%) | Carbidopa(%) |
|---|---|---|
| 0.5 | 21 | 71 |
| 1 | 33 | 87 |
| 2 | 50 | 105 |
| 3 | 62 | |
| 4 | 70 | |
| 6 | 81 | |
| 8 | 94 | |

TABLE 8
Dissolution Results for Formulation E

Cumulative Percent Release

| Time (h) | Levodopa (%) | Carbidopa(%) |
|---|---|---|
| 0.5 | 27 | 102 |
| 1 | 43 | |
| 2 | 63 | |
| 3 | 76 | |
| 4 | 85 | |
| 6 | 94 | |
| 8 | 101 | |

TABLE 9
Dissolution Results for Formulation F

Cumulative Percent Release

| Time (h) | Levodopa (%) | Carbidopa(%) |
|---|---|---|
| 0.5 | 26 | 72 |
| 1 | 40 | 95 |
| 2 | 61 | 103 |
| 3 | 72 | |
| 4 | 88 | |
| 6 | 93 | |
| 8 | 99 | |

Thus, two drugs with totally different release profiles can be delivered with independent control of the rate of release of each drug. It should be noted that this control can be achieved by shaping and sizing the core tablet, e.g. by providing it with hole of predetermined size or shape, without necessitating a change in formulation.

### Example 4

### Enterically Coated Methylphenidate Core Tablet in an Annular body Containing both Levodopa and Carbidopa

### A. Core Tablet

Methylphenidate granulate: Methylphenidate (150 grams), anhydrous lactose (420 grams), and hydroxypropylcellulose (Klucel LF™, 30 grams) were mixed in a Diosna P 1/6 high shear granulator at 380 rpm for 5 minutes. Purified water (60 grams) was added over the next minute while continuing to granulate at 380 rpm. The granulate was then massed for a further 10 seconds at the same speed. The formed granulate was dried for 30 minutes in a Diosna Mini Lab fluidized bed drier to less than 2% volatiles at an inlet temperature of 50° C. and a fan setpoint of 40%. The volatile content was tested at 105° C. using a Sartorious MA 30 LOD tester. The yield of dry granulate was 586.9 gram (98.4%).

The dried granulate was next milled using an Erweka mill with a screen of 0.8 mm. The yield of the milled granulate was 583.5 grams (99.4%).

Tableting mixture: The milled, dry, methylphenidate granulate (502.5 grams), was mixed in the dry state with Microcelac 100 USP (178.6 grams), and hydroxypropylmethylcellulose (Methocel K15M™, 193.6 grams) in a 5 liter V mixer for 5 minutes. Magnesium stearate NF/EP (5.3 grams) was added and the V mixer operated for a further half a minute. The yield of the dry mix of powders was 875.2 grams.

Tablet formation: The dry mix powder was pressed into tablets on a Kilian RTS 20 tablet press using 5 mm flat faced punches. The tablets weighed an average of 71.8 mg (design 70±3.5 mg), had a hardness of 4 Kp (design 3-6 Kp) and a tablet thickness of 2.65 mm (design 2.4-2.7 mm). The weight of the tablets produced was 676.9 grams.

Enteric coating: Purified water (522 grams) was placed in a mixing vessel. Talc (19.2 grams), and triethylcitrate (38.4 grams) were added and the mixture was stirred for 15 minutes with a magnetic stirrer. Eudragit L-30 D55™ (639.6 grams) was added and the mixture stirred gently. The coating mixture was passed through a 150μ screen and then continually mixed gently.

Methylphenidate core tablets (676.9 grams) were placed in the drum of a Hi coater perforated pan coater and heated to 30-32° C. while the drum was turning at 7 rpm. The coating mixture was sprayed onto the tablets in the perforated pan coater turning at 12 rpm with the tablet bed maintained at 30-32° C. with the inlet air temperature set at 44° C. until an average of 8 mg per tablet of enteric coat had been added to the tablets. The tablets were air dried in the drum for five minutes after the spraying was halted and subsequently dried on an aluminum tray in a drying oven set at 40° C. for 24 hours. The yield of enteric coated tablets was 729.3 grams.

### B. Annular Body

Carbidopa/levodopa granulate: Carbidopa (191.7 grams), levodopa (708.3 grams), and polyvinylpyrollidone (Povidone K-30™, 100 grams) were added to a Diosna P1/6 high shear granulator and mixed for 5 minutes at 260 rpm. Over the next minute ethanol (95%, 120 grams) was added as a granulating solvent while the mass was being mixed at 260 rpm. The mixture was then massed at 520 rpm for 45 seconds. The wet granulate was then milled through a 2.5 mm screen in an Erweka mill and subsequently dried for 35 minutes in a Diosna Mini Lab fluidized bed drier to less than 2.5% volatiles at an inlet temperature of 50° C. and a fan setpoint of 55%. The volatile content was tested at 105° C. using a Sartorious MA 30 LOD tester. The yield of dry granulate was 851.8 gram (85.2%). The dry granulate was milled once again in a Quadro Comil through an 1143μ screen to yield 820.2 grams of dried, milled granulate.

Tableting mixture: The milled, dry, carbidopa/levodopa granulate (612 grams) was placed in a 5 liter V mixer. Microcelac 100™(427.5 grams), polyethylene oxide (Polyox WSR-N-750™, 300 grams) and polyvinylpyrollidone (Povidone K-30™, 150 grams) were added and mixed in the V mixer for 5 minutes. Magnesium stearate NF/EP (10.5 grams) was added and the V mixer operated for a further half a minute. The yield of the dry mix of powders was 1493.5 grams.

Tablet formation: The enteric coated methylphenidate core tablets were added to the tablet feeder and the carbidopa/levodopa tableting mixture was added to the powder feeder of a Manesty LP39 press using the special spring loaded core rod tooling for making the annular sheathed core tablets. The lower punch was flat beveled of 11 mm diameter and an inner hole (for the core rod) of 5.5 mm diameter. The upper punch was flat beveled of 11 mm diameter with a protrusion that was 1.2 mm tall and 5.5 mm diameter with slight tapering. The final tablets so formed weighed an average of 526.9 mg (design 530±26 mg), had a hardness of 4.4 Kp (design 3-8 Kp) and a tablet thickness of 5.9 mm (design 2.4-2.7 mm). The weight of the tablets produced was 810.2 grams.

Each tablet contains 130 mg levodopa and 35 mg carbidopa in the annular body and 10 mg methylphenidate in the enterically coated core tablet.

### C. Drug release

The tablets were tested for drug release in a USP Apparatus II in 900 ml 0.1N HCl at 37° C. and 50 rpm for 3 hours and then pH=6.8 phosphate buffer for an additional 4 hours. The concentrations of methylphenidate, levodopa, and carbidopa were measured by HPLC analysis. The results are given in Table 10. One can see that the enteric coat has prevented the methylphenidate from being released for the three hours that the system is in the acidic buffer. During that time all the levodopa and carbidopa have been released. When transferring to a neutral buffer the methylphenidate is released over a few hours.

TABLE 10
Cumulative release of methylphenidate, levodopa, and carbidopa

| Time (hours) | % methylphenidate | % levodopa | % carbidopa |
|---|---|---|---|
| 1 | 0 | 40.3 | 40.0 |
| 2 | 0 | 78.1 | 77.8 |
| 3 | 0 | 97.8 | 97.7 |
| 4 | 43.3 | n.m. | n.m. |
| 5 | 74.0 | n.m. | n.m. |
| 6 | 86.7 | n.m. | n.m. |
| 7 | 89.9 | n.m. | n.m. |

n.m. = not measured

### Example 5

### Enterically Coated Rasagiline Core Tablet in a Placebo Annular Body

### A. Core Tablet

Rasagiline granulate: Rasagiline mesylate (40 grams), and Microcelac 100™ (360 grams) were mixed in a Diosna P 1/6 high shear granulator at 380 rpm for 5 minutes. Purified water (130 grams) was added over the next minute while continuing to granulate at 380 rpm. The granulate was then massed for a further 1 minute at the same speed. The formed granulate was dried for 30 minutes in a Diosna Mini Lab fluidized bed drier to less than 1.5% volatiles at an inlet temperature of 60° C. and a fan setpoint of 50%. The volatile content was tested at 105° C. using a Sartorious MA 30 LOD tester.

The dried granulate was using a Quadro Comil with a screen of 1143μ. Two sublots were produced so as to have enough material for the next stage.

Tableting mixture: The milled, dry, rasagiline granulate (558.0 grams), was mixed in the dry state with Microcelac 100 USP (2049.6 grams), and Crospovidone NF (53.7 grams) in a 5 liter V mixer for 5 minutes. Magnesium stearate NF/EP (21.5 grams) was added and the V mixer operated for a further half a minute. The yield of the dry mix of powders was 2674.7 grams.

Tablet formation: The dry mix powder was pressed into tablets on a Kilian RTS 20 tablet press using 5 mm flat beveled punches. The tablets weighed an average of 75.0 mg, had a hardness of 8.7 Kp and a tablet thickness of 2.75 mm. The weight of the tablets produced was 2238.7 grams.

Enteric coating: Purified water (1044 grams) was placed in a mixing vessel. Talc (38.4 grams), and triethylcitrate (38.4 grams) were added and the mixture was stirred for 15 minutes with a magnetic stirrer. Eudragit L-30 D55™ (1279.2 grams) was added and the mixture stirred gently. The coating mixture was passed through a 150μ screen and then continually mixed gently.

Rasagiline core tablets (2238.7 grams) were placed in the drum of a Hi coater perforated pan coater and heated to 28-30° C. while the drum was turning at 7 rpm. The coating mixture was sprayed onto the tablets in the perforated pan coater turning at 12 rpm with the tablet bed maintained at 28-30° C. with the inlet air temperature set at 60° C. until an average of 6.5 mg per tablet of enteric coat had been added to the tablets. The tablets were air dried in the drum for five minutes after the spraying was halted and subsequently dried on an aluminum tray in a drying oven set at 40° C. for 24 hours.

### B. Annular Body

Tableting mixture: Polyethylene oxide (Polyox WSR-N-750™, 600 grams), Microcelac 100™(486 grams), ethylcellulose (Ethocel 7 cps, 600 grams), and polyvinylpyrollidone (Povidone K-30™, 300 grams) were placed in a 5 liter V mixer and mixed for 5 minutes. Magnesium stearate NF/EP (14 grams) was added and the V mixer operated for a further half a minute. The yield of the dry mix of powders was 1990.1 grams.

Tablet formation: The enterically coated rasagiline core tablets were added to the tablet feeder and the tableting mixture was added to the powder feeder of a Manesty LP39 press using the special spring loaded core rod tooling for making the annular sheathed tablets. The lower punch was flat beveled of 9 mm diameter and an inner hole (for the core rod) of 5 mm diameter. The upper punch was flat beveled of 9 mm diameter with a protrusion that was 1.2 mm tall and 5 mm diameter with slight tapering. The final tablets so formed weighed an average of 310 mg, had a hardness of 6.4 Kp and a tablet thickness of 5.4 mm.

Each tablet contained the equivalent of 1 mg rasagiline as the mesylate salt in the enterically coated core tablet.

### C. Drug Release

The tablets were tested for drug release in a USP Apparatus II in 500 ml 0.1N HCl at 37° C. and 50 rpm for 3 hours and then pH=6.8 phosphate buffer for an additional 2 hours. The concentration of rasagiline was measured by HPLC analysis. The results are given in Table 11. In parallel, the enterically coated core tablets before their insertion into the annular body were also tested. These results are also given in Table 11. One can see that the enteric coating has prevented the rasagiline from being released for the three hours that the system is in the acidic buffer in both cases. When transferring to a neutral buffer the rasagiline is released in an immediate fashion. The annular body has not damaged the enteric coating and its performance and will protect the enteric coating against mechanical forces in the gastrointestinal tract.

TABLE 11
Cumulative Rasagiline release

| | Core tablet | Core tablet |
|---|---|---|
| | within annular | without annular |
| | body | body |
| Time (hours) | % rasagiline | % rasagiline |
| 1 | 0 | 0 |
| 2 | 0 | 0 |
| 3 | 0 | 0 |
| 3.15 | 31 | 27 |
| 3.5 | 89 | 80 |
| 3.75 | 101 | 90.5 |
| 4 | 104 | 93.5 |
| 5 | 107 | 97.9 |

Having thus described the invention with reference to certain preferred embodiments, other embodiments will be apparent from this description to those skilled in the art to which the invention pertains. It is intended that the specification is considered exemplary only, with the scope and spirit of the invention being indicated by the claims which follow.

What is claimed is:

1. A pharmaceutical dosage form for oral administration to a patient comprising an enterically coated core tablet containing an active pharmaceutical ingredient sheathed in an annular body of compressed powder or granular material.

2. The pharmaceutical dosage form of claim 1, wherein the active pharmaceutical ingredient is selected from the group consisting of methylphenidate, rasagiline, carbidopa, levodopa, and pharmaceutically acceptable salts and solvates thereof.

3. The pharmaceutical dosage form of claim 2, wherein the active pharmaceutical ingredient is methylphenidate or rasagiline.

4. The pharmaceutical dosage form of claim 1, wherein the core tablet further contains one or more excipients selected from the group consisting of anhydrous lactose, hydroxypropylcellulose, microcrystalline cellulose, hydroxypropylmethylcellulose, and crospovidone.

5. The pharmaceutical dosage form of claim 1, wherein the annular body further contain one or more excipients from the group consisting of polyvinylpyrollidone, microcrystalline cellulose, polyethylene oxide, and ethylcellulose.

6. The pharmaceutical dosage form of claim 1, wherein the enteric coating prevents release of the active pharmaceutical ingredient in the stomach and allows release of the active pharmaceutical ingredient in the small intestine.

7. The pharmaceutical dosage form of claim 1, wherein substantially none of the active pharmaceutical ingredient is released in at least two hours when release is measured in a United States Pharmacopeia apparatus II in either 900 ml or 500 ml of 0.1 N hydrochloric acid at 37° C. with a stirring rate of 50 revolutions per minute.

8. The pharmaceutical dosage form of claim 1, wherein substantially none of the active pharmaceutical ingredient is released in three hours when release is measured in a United States Pharmacopoeia apparatus II in either 900 ml or 500 ml of 0.1 N hydrochloric acid at 37° C. with a stirring rate of 50 revolutions per minute.

9. The pharmaceutical dosage form of claim 8 wherein substantially all of the active pharmaceutical ingredient is released at pH≧7.

10. The pharmaceutical dosage form of claim 1 wherein the rate of release of the active pharmaceutical ingredient from the dosage form is substantially equal to the rate of release from a core table alone, before sheathing, when each is independently measured in a United States Pharmacopeia apparatus II at 37° C. and 50 revolutions per minute.

11. A method of treating a patient suffering from Parkinson's disease and presenting impaired gastric motility comprising the step of administering to the patient a dosage form of claim 1, comprising an enterically coated core table comprising methylphenidate sheathed in an annular body of compressed powder or granular material and comprising levodopa, carbidopa, or both.

12. A pharmaceutical dosage form for co-administration of methylphenidate and either or both of lovodopa and carbidopa to a patient comprising an enterically coated core table comprising methylphenidate sheathed in an annular body of compressed powder or granular material and comprising levodopa, carbidopa, or both.
